(19) United States
(12) Patent Application Publication
(10) Pub. No.: US 2004/0033573 A1
(43) Pub. Date: Feb. 19, 2004

(54) Method for producing lactic acid

(76) Inventors: Birgir Norddahl, Ringe (DK); Soren Eriksen, Odense (DK); Frederik Moller Pedersen, Odense (DK)

Correspondence Address: HARNESS, DICKEY & PIERCE, P.L.C., P.O. BOX 8910, RESTON, VA, 20195, US

(21) Appl. No.: 10/297,090

(22) PCT Filed: May 30, 2001
(86) PCT No.: PCT/DK01/00375

(30) Foreign Application Priority Data
May 30, 2000 (DK) PA 2000-00851

(51) Int. Cl. C12P 7/56

(52) U.S. Cl. 435/139

(57) ABSTRACT

A method for producing lactic acid, comprising producing lactic acid from a sugar-containing fermentation liquid in a fermentor by means of lactic acid-forming bacteria to result in a lactate salt, typically ammonium, sodium or potassium lactate, and isolating lactic acid by subjecting the fermented fermentation liquid to a first ultrafiltration step to result in a substantially polymer-free permeate comprising at least one lactate salt, acidifying the permeate to a pH value of below about 3.9, performing at least one additional isolation step in which the acidified permeate is subjected to nanofiltration and/or reverse osmosis, and preferably subjecting the resulting product to electrodialysis using bipolar electrodialysis membranes. Fermentation is preferably performed using whey protein as a nutrient substrate and by adding at least one protein-hydrolysing enzyme directly to the fermentor during the fermentation process so that hydrolysis of protein to amino acids takes place simultaneously with the fermentation of sugar into organic acid.

### FIELD OF THE INVENTION

The present invention relates to a process for the fermentative production of lactic acid and for the isolation of lactic acid from a lactic acid-containing solution.

### BACKGROUND OF THE INVENTION

European patent No. 230.021 describes a process in which glucose is fermented continuously to lactate, after which lactic acid is extracted from the solution by means of electrodialysis, where pH in the fermentor is controlled by removing the lactic acid at the same rate as the rate at which it is formed, the contents of the fermentor being recirculated over the electrodialysis unit. Yeast extract and inorganic salts are used as nutrients. A disadvantage of this system is that bacteria in the fermentor liquid are known to adsorb to the electrodialysis membranes, causing the electrical resistance in the electrodialysis unit to increase, which results in a substantially increased power consumption for the electrodialysis process.

Boyaval et al. (Biotechnology Letters Vol. 9, No. 3, 207-212, 1987) describe a bioreactor for lactic acid fermentation using a three-stage fermentation process that includes the production of biomass and lactic acid in the first stage, separation and concentration of the cells by ultrafiltration in the second stage, and lactate concentration and purification by electrodialysis in the third stage. It is reported, however, that this system exhibits the disadvantage of dogging of the ultrafiltration membranes, resulting in drastic restriction of permeate flow.

U.S. Pat. No. 4,110,175 also describes a general method for electrolytic purification of organic acids, including lactic acid. An improved version of this method is described in U.S. Pat. No. 5,002,881, in which lactic acid is formed as ammonium lactate through fermentation of a glucose-containing medium, which makes it possible to use ultrafiltration to separate the ammonium lactate from the fermentation liquid, as the retentate from the ultrafilter is returned to the fermentor. In this way there is no adsorption of bacteria to the membran s in the subsequent electrodialysis processes, and power consumption is therefore lower. The micro-organism used in the patent is Bacillus coagulans, which has the property of not needing any special nutrient medium containing y ast extract r corn steep liquor, which are otherwise known to be necessary to maintain lactic acid fermentation when lactic acid bacteria are used. Prior to electrodialysis, the fermentor liquid is concentrated by means of reverse osmosis (RO), and the concentrated liquid is subsequently treated in an electrodialysis unit in which lactic acid is formed from ammonium lactate by means of bipolar membranes in a single operation. In this operation ammonium hydroxide is formed at the same time and can be returned to the fermentor as a medium for neutralisation of lactic acid. In this process, however, amino acids are used as a nutrient for the fermenting bacteria, which results in the disadvantage of relatively high costs. A further disadvantage is that RO used for concentration will result in non-converted organic matter (residual glucose and amino acids) being included in the electrodialysis treatment with bipolar membranes, where they contribute to reducing the process efficiency. Also, the resulting product might not be heat-stable due to the presence of residual sugars in the lactic acid.

The formation of amino acids from whey proteins and the use of whey protein as a nutrient in the fermentation of lactose in whey is described in U.S. Pat. No. 4,698,303. However, U.S. Pat. No. 4,698,303 has the disadvantage of requiring an independent hydrolysis for the production of amino acids from whey protein, the hydrolysis being carried out as a separate acidic enzymatic process, after which the hydrolysed product is fed to the membrane fermentor as a nutrient.

U.S. Pat. No. 5,503,750 describes a method for recovering lactate salts using a combination of ultrafiltration, nanofiltration and reverse osmosis. The overall recovery of lactic acid disclosed therein is rather low (not more than about 54%).

WO 98/28433 discloses a method for fermentation of lactic acid using whey protein by adding a protein-hydrolysing enzyme to the fermentor during the fermentation so that hydrolysis of protein to amino acids takes place simultaneously with the fermentation of sugar into organic acid, and isolating lactic acid resulting from the fermentation using an ultrafiltration step and subsequently at least two electrodialysis steps.

The purification procedure described in WO 98/28433 has different disadvantages e.g. with respect to the consumption of chemicals. Thus, ion exchange columns utilize chemicals in the form of inorganic acids and bases for regeneration, which cannot be recovered for reuse. Also the regeneration procedures results in a loss of lactic acid as the columns are flushed with the reg neration solutions. Removing bivalent ions on chelating ionexchange furthermore requires a precise method to monitor break-through if contamination of the subsequent bipolar electrodialysis is to be avoided.

In WO 98/28433 the lactic acid is transported across membranes in both conventional and bipolar electrodialysis with the use of electrical energy. The use of electrical energy may represent a significant contribution to the production price of the lactic acid. Furthermore, the recovery in the conventional electrodialysis is quite low, especially if an acceptable power efficiency is desired.

The present invention is a further development based on the invention disclosed in WO 98/28433 and provides a novel purification procedure for isolation of lactic acid which has the advantage of being simple and inexpensive and resulting in a high lactic acid recovery rate requiring fewer steps than the above known methods.

Additionally, it has surprisingly been found by the present inventors, that nanofiltration and/or reverse osmosis may be used as an efficient alternative to conventional electrodialysis for the removal of sugar and proteins, and as an alternative to chelating ion exchange for the removal of bivalent ions such as calcium and magnesium ions. Furthermore, it has been found that electrodialysis can advantageously be used as an additional step after a nanofiltration and/or reverse osmosis step to remove the remaining inorganic ions, which therefore eliminates the need for further polishing on ion-exchange.

### BRIEF DISCLOSURE OF THE INVENTION

It is an object of the present invention to provide a process by which lactic acid can be produced and isolated in a simple and inexpensive manner, and in particular to provide a new and improved isolation method for organic acids such as lactic acid.

The invention thus relates to a method for producing lactic acid, comprising producing lactic acid from a sugar-containing fermentation liquid in a fermentor by means of lactic acid-f rming bacteria to result in a lactate salt, and isolating lactic acid by subj cting th fermented fermentation liquid to a first ultrafiltration step to result in a substantially polymer-free permeate comprising at least one lactate salt, acidifying th permeate to a pH value of below about 3.9, and performing at least one additional isolation step in which the acidified permeate is subjected to nanofiltration and/or reverse osmosis. Finally, inorganic salts are typically removed by electrodialysis.

A further aspect of the invention relates to a method for isolating organic acid from a solution containing an organic acid salt

As mentioned above, the present invention has the advantage of being simple and inexpensive implying fewer steps than presently known methods and resulting in a high lactic acid recovery rate. Using the invention, it will typically be possible to reach an overall recovery rate of about 90-95%, or even higher, such as about 96-98% or more, based on the amount of sugar added to the fermentor. Further, a number of additional advantages are obtained by means of the invention, including:

avoidance of the need to use chemicals to regenerate ion exchange materials, thereby avoiding waste streams in the form of acids and bases from this regeneration;

a higher operating efficiency, since in contrast to a process using ion exchange, there is no risk of calcium or magnesium ions passing through the ion exchange resins; the process is therefore also easier to control;

all of the effluent streams are recycled, the acids and bases generated in the optional bipolar electrodialysis step being returned to the process; and

a reduction in the amount of waste products, since the only “waste” that is generated is in the concentrate from nanofiltration, which contains Ca/Mg ions and coloured compounds.

### DETAILED DESCRIPTION OF THE INVENTION

According to the invention, lactic acid is produced by fermentation, typically fermentation of a sterilised growth medium comprising a sugar-containing solution and a protein, e.g. whey protein in the form of whey permeate from production of whey protein concentrate. Fermentation is preferably performed by adding to the fermentor one or more protein-hydrolysing enzymes, in the following called proteases, to result in continuous production of hydrolysed protein simultaneously with ferm ntation by m ans of a bacteria culture that produces lactic acid, .g. as disclosed in the above-mentioned WO 98/28433.

Whey protein is a well-known protein mixture derived from milk and consisting mainly of β-lactoglobulin, α-lactalbumin, bovine serum albumin and immunoglobulins. It is described in numerous places in the literature, e.g. in Mulvihill, D. M. & Donovan, M.: “Whey proteins and their thermal denaturation—A review”, Irish Journal of Food Science and Technology, 11, 1987, pp. 43-75, to name one example.

While the present invention preferably uses whey protein due to the fact that it is readily available and relatively inexpensive, any suitable protein source may be used in the process of the invention, for example yeast extract, corn steep liquor, malt sprout extract or casein hydrolysates. Of course, a mixture of different types of proteins may also be used. Regardless of the protein source, the proteins may be hydrolysed to amino acids by any suitable protease to provide nutrients for the fermentation. Many such proteases are commercially available, an example of which is Flavourzyme®, which is available from Novo Nordisk A/S, Denmark. As the lactic acid-forming bacteria, any suitable lactic acid-forming bacteria, or a combination of more than one lactic acid bacteria, may be used, e.g. a bacteria of the genus Lactobacillus, such as L. helveticus, L. delbrueckii, L. casei, L. acidophilus or L. bulgaricus. The lactic acid-forming bacteria such as Lactobacillus sp. may be used alone or together with another micro-organism, for example as a co-culture with e.g. Streptococcus thermophilus.

The use of different strains of a lactic acid bacteria such as L. helveticus makes it possible to form L(+), L(−) or D(−) as well as mixtures of L(+)/(−) and D(−). In the following, the term “lactic acid” is intended to refer to any one of these types of lactic acid or mixtures thereof.

As indicated above, the enzyme is preferably added directly to the fermentor. This allows fermentation and hydrolysis to take place in the same container, i.e. the fermentor, which results in a simple and inexpensive fermentation process. Advantageously, ultrafiltration membranes may be coupled to the fermentor without being fouled by protein, as the hydrolysis using direct addition of enzyme to the fermentor is so quick that the proteins are hydrolysed down to peptides and amino acids before any substantial protein deposits can occur.

A further advantag of using direct addition of enzymes to the f rmentor is that it makes it possible to us an ultrafilter with a very small pore siz, e.g. not more than about 10,000 Dalton and preferably lower. It is thus possible to maintain a constant high flux with an ultrafilter having a cut-off value of e.g. about 5,000 Dalton, so that purification of the fermentation product, the lactic acid, can be simplified, as the content of higher polymeric constituents (mainly unhydrolysed proteins, polyglucans and other polysaccharides created by the lactic acid bacteria) in the permeate from the ultrafilter coupled to the fermentor is lower than in other known systems. Finally, the use of ultrafiltration in connection with the fermentation means that the added enzymes will stay in the fermentor, as they are unable to pass through the membrane, so that the duration of action of the enzymes is longer, which makes it possible to obtain substantial savings on the consumption of enzymes as compared to other lactic acid fermentation systems.

The “sugar” in the sugar-containing solution used according to the present invention can be any suitable sugar for lactic acid fermentation, for example a monosaccharide such as glucose, fructose or galactose, a disaccharide such as sucrose, maltose, cellobiose or lactose, or a polysaccharide. A mixture of different sugars can of course also be used. The sugar may suitably be derived e.g. from a whey permeate, but it may also be derived from any other source.

In a preferred embodiment, the pH in the fermentation liquid is kept substantially constant within the range of about pH 5-7 by addition of a suitable base. The base may e.g. be ammonia, typically in the form of ammonia gas, or NaOH, KOH or a mixture thereof (in the following designated as “Na/KOH”), all of which form water-soluble salts with lactic acid. The use of ammonia as the base has the advantage that it provides a source of nitrogen for the lactic acid bacteria compared to other bases. Furthermore, ammonia is less expensive than many other bases. Na/KOH is, however, easier to recover in the subsequent purification of the lactic acid because the volatile nature of ammonia results in considerable loss to the surroundings and undesirable diffusion through the membranes used in the isolation of the lactic acid.

After fermentation, the fermentation liquid is as indicated above subjected to an ultrafiltration process which retains the retentate containing bacteria culture and non-hydrolysed protein, and allows dissolved matter to pass, including lactic acid formed in the fermentation process. The lactic acid may e.g. be in the form of ammonium lactate when ammonia is added as a base or sodium or potassium lactate when Na/KOH is added. The result is a substantially polymer-free perm ate comprising at least one lactate salt. In the present context the term “polymer-free” is intended to include unhydrolysed proteins, polyglucans and other polysaccharides created by the lactic add bacteria and bacterial biomass.

The permeate from the ultrafiltration process is then acidified by addition of a suitable acid. Although the nature of the acid is not believed to be critical, and the use of either an inorganic or an organic acid is contemplated, acidification preferably takes place using an inorganic acid, for example hydrochloric acid, e.g. in the form of concentrated hydrochloric acid such as hydrochloric add having a concentration of about 20-40%, such as about 30%.

The acidification comprises adjustment of the pH to a value of below about 3.9, in particular to below the pKa-value of lactic acid (3.86), typically below about 3.8, preferably to a pH below about 3.5, and more preferably between about 2.5 and 3.0. As a result, the free lactate ions will combine with hydrogen ions to form lactic add having no net electrical charge. Free ions in the solution will thus comprise those of the inorganic acid used for acidification of the ultrafiltration permeate, e.g. chloride ions, and the base used for neutralisation, e.g. ammonia or Na/KOH, as well as trace amounts of other salts that happen to be present.

The resulting acidic solution is then typically subjected to a nanofiltration process, in particular using a nanofiltration membrane with the ability to retain divalently charged ions, and molecules larger than about 180 g/mol. Ions with a single charge are only partly retained, while small uncharged molecules permeate the membrane freely.

Lactic acid, being uncharged at the low pH of the acidic solution, therefore permeates the membrane while calcium and magnesium ions are retained together with larger molecules, e.g. residual sugars, proteins and coloured compounds.

The resulting permeate is therefore free of calcium and magnesium, thereby preventing precipitation of salts, for example calcium salts such as calcium phosphate that might otherwis l ad to a slow irreversible scaling of the membranes in a subsequent electrodialysis treatment of the permeat. Moreov r, since th nanofiltration membrane retains compounds that otherwise would colour the solution, the col ur in the permeat is reduced significantly.

The permeate will at this point, however, also contain most of the inorganic acid added prior to nanofiltration as well as the neutralising agent, e.g. ammonium or Na/KOH, because the reject of these salts is low at the reduced pH.

As an alternative to the nanofiltration membrane, a reverse osmosis membrane can be used. This results in a more pure lactic acid permeate, i.e. containing fewer undesired ions, but it has the disadvantage of lower capacity.

A second alternative is filtering the acidified ultrafiltration permeate twice (or, if desired, more than two times) by nanofiltration to further reduce the concentration of calcium, magnesium and/or coloured compounds if necessary or advantageous. Since the concentration of divalently charged ions and membrane fouling compounds in the feed to the second nanofiltration is relatively low, a high capacity and recovery is expected. Therefore, adding a further (third) nanofiltration step is expected have very little effect on the overall recovery. Such further filtration steps may also, as described above, be performed by the use of reverse osmosis.

In the case where more than one nanofiltration and/or reverse osmosis step are applied it may be advantageous to increase the concentration of the lactic acid in the permeate from the first nanofiltration or reverse osmosis by partial evaporation before performing the subsequent filtration. The temperatures achieved during evaporation will bring residual protein and sugar, however in very small amounts, to react and form coloured Malliard compounds, which are then removed in the second nanofiltration or reverse osmosis. The lactic acid containing permeate is concentrated to between 5 and 90%, including between 10 and 50% such as to about 20%.

Reducing protein and sugar at this point minimises fouling from these components in a subsequent bipolar electrodialyser and prevents or minimises formation of coloured components in the final concentration of the lactic acid.

It will be apparent to persons skilled in the art that a number of diff rent nanofiltration membranes with different por sizes ar commercially availabl, and persons skilled in th art will b able to determine a suitable pore size to obtain the d sired purification using such commercially availabl nanofiltration membranes. While not wishing to be bound by any particular theory, it is believed, however, that the purification obtained by nanofiltration may be more a result of transport of lactic acid through the membrane due to its neutral charge at the acidic pH rather than a filtration effect based on size. It is therefore believed that pore size of the nanofiltration membrane is not critical.

The pore size for both nanofiltration and reverse osmosis is defined in Mulder, M.: “Basic Principles of Membrane Technology”, 2nd edition, 1998, as being less than 2 nm. As noted above, reverse osmosis has a greater ability to retain undesired ions (Ca/Mg) than nanofiltration, but a lower flux. It will be apparent to persons skilled in the art that it is possible to utilise a variety of different combinations of nanofiltration and/or reverse osmosis membranes in order to obtain the desired purification in any given situation.

Subsequent hereto, the permeate from nanofiltration or reverse osmosis is preferably subjected to an electrodialysis process in which ion-selective and bipolar membranes separate the inorganic salts from the lactic acid. Lactic acid will at the feed pH of e.g. about 2.5-3.0 have no electrical charge and will thus not be transported in the electrical field during electrodialysis. Chloride ions and the base (ammonium or Na/KOH) will on the other hand be transported in the electrical field.

Lactic acid is thus recovered in the feed stream, which is deionised during electrodialysis.

The advantage of this electrodialysis procedure is that chloride is transported in the electrical field rather than lactate. The mobility of chloride in the electrodialysis membranes is much higher than the mobility of lactate and thus a much larger power efficiency is achieved. Also, the need for a “polishing” ion removal step is avoided or at least significantly reduced, since all or at least almost all ions are recovered either in the base compartment or the acid compartment in the case of bipolar electrodialysis. Furthermore, loss of lactic acid is avoided since all streams are recycled.

Various arrangements are possible for the electrodialysis. For example, the bipolar electrodialysis can be operated using a three-compartment configuration, i.e. with separat compartments for brine, base and acid containing streams. Th brin compartment, to which the lactate is fed, is passed through the membrane stack in the space between the monopolar cationic and anionic membranes. The base stream is led between the monopolar cationic membrane and the anionic side of the bipolar membrane, where the hydroxide ions are generated. The acid stream is led between the monopolar anionic membrane and the cationic side of the bipolar membrane, where acid is generated.

Thus the anions (mainly chloride) will be transported from the brine compartment, through the monopolar anionic membrane, to the acid compartment, where they combine with protons generated by the bipolar membrane to form the corresponding acid. Similarly, cations (Na, K or NH₄⁺) are transported from the brine compartment, through the monopolar cationic membrane, to the base compartment, where they combine with hydroxide ions generated by the bipolar membrane to form bases. In this way, hydrochloric acid (or other acid) and Na/K hydroxide (or other base) can be recovered in the acid and base compartments, respectively.

Alternatively, the bipolar electrodialysis can be operated using a two-compartment configuration, where either the cationic or the anionic monopolar membranes are omitted. In this mode of operation, only anions or cations are removed from the feed compartment and replaced with either protons or hydroxide ions. A brine compartment is therefore not present in this configuration. A disadvantage of this configuration, however, is that the lactic acid-containing stream is only partly deionised, since only cations or anions are removed.

Finally, the deionisation can be performed with conventional electrodialysis using only monopolar membranes. In this configuration, the lactic acid containing stream is deionised as in the three-compartment bipolar electrodialysis. Cations and anions are, however, recovered in single common stream and not as separate acid and base streams.

Thus, performing the electrodialysis with a three-compartment bipolar electrodialysis is thought to be most advantageous approach, although the invention is not limited hereto. Regardless of the exact electrodialysis arrangement chosen, the invention has the important advantage that lactate is not transferred from one compartment to another, but rather is deionised in the electrodialysis step.

The ammonium or Na/K hydroxide-containing solution that is recovered during three-compartment electrodialysis is then typically led back to th r actor in an amount that regulates pH to the set value, e.g. a pH in the range of about 5.0-7.0, preferably about 5.5-6.5, such as about 5.5-6.0. The hydrochloric acid recovered in the acid compartment is recycled for pH adjustment in the ultrafiltration permeate prior to nanofiltration, e.g. to a pH in the range of about 2.5 to 3.0.

As an alternative to recovering hydrochloric acid in the acid compartment in the bipolar electrodialysis in water, ultrafiltration permeate from the fermentor may be recycled in the acid compartment. In this way the ultrafiltration permeate is acidified in the bipolar electrodialysis rather than by addition of aqueous hydrochloric acid. This eliminates the need to concentrate the hydrochloric acid otherwise generated during electrodialysis. The calcium-containing ultrafiltration permeate can be treated in the acid compartment of the bipolar electrodialysis since no precipitation is expected to take place under the acidic conditions therein.

Although the procedure for isolation of lactic acid according to the present invention preferably comprises a combination of the above-described steps, i.e. ultrafiltration, at least one nanofiltration or reverse osmosis step, and bipolar electrodialysis, and preferably in the order described, it will be dear to persons skilled in the art that one or more steps in this procedure may, if desired or advantageous, be eliminated in certain cases, and/or the order of the steps may in certain cases be varied.

Finally, the lactic acid is purified and concentrated to the desired concentration, for example by evaporation using a falling film multi-stage vacuum evaporator. Concentration of the lactic acid may alternatively be performed by other known methods, e.g. in a compression evaporator in which any formic acid and acetic acid are distilled off together with water. Thus, the concentration of lactic add may e.g. be increased to about 50-99%, including about 60-95%, such as about 70-90%.

Afterwards or at any desired point during concentration, possible residual colour may be removed using e.g. activated charcoal or an additional nanofiltration step.

As will be apparent from the discussion above, th method presented herein is useful for the production of lactic acid. How v r, it is contemplated that th method for isolation of lactic acid may also be advantageously applied for the isolation of organic acids in general.

Thus, in a further aspect of the present invention there is provided a method for isolating an organic acid from a solution containing an organic acid salt, comprising the steps of:

i) forming a substantially polymer-free permeate containing the organic acid salt,

ii) acidifying the permeate to a pH value of below about the pKa-value of the organic acid,

iii) subjecting the acidified permeate to at least one nanofiltration and/or reverse osmosis step to result in a organic acid-containing product,

iv) subjecting the product to an electrodialysis step,

v) concentrating the product of the electrodialysis to result in concentrated organic acid, and optionally

vi) polishing the concentrated organic acid, e.g. using nanofiltration or activated charcoal.

In accordance with the invention, the organic acid to be isolated may be any suitable carboxylic acid. Thus, as examples, the organic acid may be formic acid, acetic acid, lactic acid, butyric acid, propionic acid, valeric acid, isovaleric acid, capronic acid, heptanoic acid, octanic acid, oxalic acid, maloic acid, glutaric acid, adipic acid, glycolic acid, glycinic acid, acrylic acid, tartaric acid, fumaric acid, benzoic acid, maleric acid, phthalic acid, or salicylic acid.

The pKa-value indicates the acidity constant for the organic acid. As examples, the acidity constants of formic acid and acetic acid has been found to be 3.75 and 4.75 (measured at 20° C.), respectively.

The invention will be further illustrated in the following non-limiting examples.

### EXAMPLES

Fermentation

Lactic acid fermentation was carried out in a 100 l membrane reactor, using a Koch S4-HFK-131 spiral-wound membrane. The cut-off value of the ultrafiltration membrane was 5 kD (kiloDalton), and th total membrane area was 7.3 m². Inlet and outlet pr ssures on the m mbrane w re 4.4 and 2.9 bar, respectively.

90 l of an aqueous gr wth medium was made up on the basis of sweet whey, whey protein concentrate and additional nutrients, the composition of the medium being as follows:

| | |
|---|---|
| 9.5% by weight of | whey protein |
| 4.0% by weight of | lactose |
| 1.5% by weight of | yeast extract |
| 0.3% by weight of | K₂HPO₄ |
| 0.04% by weight of | MgSO₄, 7 H₂O |
| 0.015% by weight of | MnSO₄, 4 H₂O |
| 0.1% by weight of | Tween ® 80 |
| 0.006% by weight of | cysteine hydrochloride |

The medium was heated to 70° C. for 45 min and cooled to the fermentation temperature of 45° C. 18 g of freeze-dried Lactobacillus helveticus culture and 53 g of Flavourzyme® enzyme were added. Fermentation was carried out batchwise under anaerobic conditions for 9 hours. The continuous fermentation was then started. The aqueous feed medium was based on whey permeate and had the following composition:

| | |
|---|---|
| 0.35% by weight of | whey protein |
| 0.01% by weight of | Flavourzyme ® |
| 4.0% by weight of | lactose |

The pH in the reactor was adjusted to 5.75 with ammonia gas.

The biomass concentration was kept at approx. 7-8% via a continuous bleed of reactor content. With this biomass concentration, the permeate flux on the ultrafilter was constant during the fermentation and approx. 1 l/min (8.2 l/(m²*h)). No cleaning-in-place was done on the ultrafilter during 34 days of continuous fermentation.

The dilution rate (D) in the fermentor was varied between 0.15 and 0.3 h⁻¹. This had no effect on the conversion yield, which was constant at 99.5% or mor during the 34 days of ferm ntation. The lactate concentration in the ultrafiltration permeate was 4.0%, and the productivity at D=0.3 h⁻¹ was 12 g/(l*h).

Further isolation of lactic acid after ultrafiltration was performed using membrane filtration, bipolar electrodialysis, evaporation and “polishing” using activated charcoal as described below.

### Example 1

In this example, the ultrafiltration permeate was treated on a Labstak M20 (from DSS, Nakskov, Denmark) fitted with NF45 nanofiltration membranes (also from DSS). The pH in the ultrafiltration permeate was adjusted with 37% technical grade hydrochloric add to a range of pH values between 5.8 and 1.97. The permeate was hereafter fed to the Labstak at 30° C., 15 bar and 7.7 l/min. It was found that the transport of lactic acid across the membrane and the reject of calcium increased with decreasing pH. The flux had a maximum at pH approx. 4.5.

For example, at feed pH 3.04 a flux of 24.51 l/m²*h was measured. Lactic acid concentration in the feed and the permeate was 35.38 g/l and 27.24 g/l respectively. Calcium concentrations were 256 ppm and 0.1 ppm in the same streams, i.e. the reject was more than 99.9%. The permeate was virtually colourless.

### Example 2

In this example, approx. 800 l of ultrafiltration permeate was adjusted with 30% technical grade hydrochloric acid to pH approx. 3 and treated on a 2.5×40 inch (6.4×102 cm) spiral wound NF45 membrane element from Filmtec Corporation. The element was fitted in a custom-made test bench from Envig Pty Ltd., South Africa. From the 800 l feed, 770 l was recovered as permeate, corresponding to more than 96% recovery. Approx. 7% of the lactic acid was lost, based on mass balance. The permeate was significantly less coloured than the ultrafiltration permeate. Calcium concentration was reduced from 259 ppm in the ultrafiltration permeate to approx. 30 ppm in the accumulated NF permeate. At 25 bar and 30° C. the flux varied from 60.92 l/m²*h initially to 14.26 l/m²*h at 96% recovery. Subsequ ntly, the calcium concentration in the NF-permeat was r duced furth r, to approx. 0.1 ppm, in a second nanofiltration.

Electrodialysis was performed on a EUR2-C-BIP stack from Eurodia Industrie SA, France. The stack was operated in a three-compartment mode with 10 cell pairs. The membranes were from Tokuyama Corporation, Japan; cationic membrane: CMX; anionic membrane: AMX; bipolar membrane: BP-1. Electrical power was supplied to the stack by a power supply from Eurodia Industri SA, France. The stack was fed via 3 pumps from 3 tanks of 6 l each.

In one example, 6 l of NF-treated ultrafiltration permeate, as described above, was added to the brine tank. 6 l of demineralised water was added to the base and acid tanks. The stack was then operated at a constant voltage drop of 80V. Samples were taken regularly from the brine tank. To these samples a concentrated solution of AgNO₃ was added in order to precipitate residual chlorine ions as AgCl. The electrodialysis was stopped when no further precipitation was seen.

The concentration of salts in the brine (the lactic acid) was reduced as follows:

| | |
|---|---|
| Cl⁻: | 99.9% |
| SO₄²⁻: | 79.1% |
| NO₃⁻: | 60.7% |
| PO₄³⁻: | 88.1% |
| Na⁺: | 98.9% |
| NH₄⁺: | 99.8% |
| K⁺: | 99.8% |
| Mg²⁺: | Not present |
| Ca²⁺: | 97.1% |

The lactic acid concentration was slightly reduced but no measurements were made. Subsequently, the brine tank was emptied, the content was collected for further purification, and another 6 l of NF permeate was added to the tank. The content of the base and acid tank was not changed. The stack was then again operated at 80V until no precipitation with AgNO₃ was seen. The reduction in ion concentration was as reported earlier, and a hydrochloric acid concentration of 4.3% was achieved in the acid tank.

In another example, the demineralised water in the acid tank was replaced by ultrafiltration permeate at a pH of 5.6. The brine tank was again filled with NF-treated ultrafiltration permeate and the base tank with demineralised water. The length of the run was determined by a test for precipitation with AgNO₃. Again the reduction in salt concentration was measured, the results being as follows:

| | |
|---|---|
| Cl⁻: | 99.8% |
| SO₄²⁻: | 72.2% |
| NO₃⁻: | App. 60% |
| PO₄³⁻: | 32.8% |
| Na⁺: | 98.5% |
| NH₄⁺: | 99.5% |
| K⁺: | 99.5% |
| Mg²⁺: | Not present |
| Ca²⁺: | Not present |

The lactic acid concentration was reduced approx. 44%.

In the acid tank, initially containing ultrafiltration permeate, pH was lowered from 5.62 to 3.17. The lactic acid lost from the brine tank was transferred to the acid tank, increasing the lactic acid concentration herein from 51.9 to 59.8 g/l.

After desalination using bipolar electrodialysis, the lactic acid was concentrated by vacuum evaporation to approx. 90%. The temperature during evaporation caused some colouring of the lactic acid. This was reduced by stirring 100 ml of 90% lactic acid with 8 g of activated charcoal (Ref. no. FE90416b from F. Zwicky, Copenhagen, Denmark) for 24 h and filtering on a 5 μm filter disk. The product was a slightly yellow, heat-stable, 90% lactic acid (where heat-stability is defined as being able to be heated at 180° under reflux for 20 min without any significant change in colour).

1. A method for producing lactic acid, comprising producing lactic add from a sugar-containing fermentation liquid in a fermentor by means of lactic acid-forming bacteria to result in a lactate salt, and isolating lactic add by subjecting the fermented fermentation liquid to an ultrafiltration step to result in a substantially polymer-free permeate comprising at least one lactate salt, acidifying the permeate to a pH value of below about 3.9, and performing at least one additional isolation step in which the acidified permeate is subjected to a nanofiltration step and/or a reverse osmosis.

2. A method according to claim 1, wherein the pH of the fermentation liquid is maintained at a substantially constant level during fermentation, preferably in the range of about 5-7, by addition to the fermentation liquid of at least one base selected from ammonia, NaOH and KOH, and mixtures thereof, whereby a lactate salt of ammonium, sodium and/or potassium is formed in the fermentation liquid.

3. A method according to claim 1, wherein the ultrafiltration step comprises ultrafiltration using a filter with a cut-off point value that prevents passage through said filter of enzymes or non-hydrolysed proteins present in the fermentation liquid, e.g. a cut-off value of not more than about 10,000 Dalton, such as about 5,000 Dalton.

4. A method according to claim 1, wherein the permeate from ultrafiltration is acidified to a pH of below about 3.8, preferably below about 3.5, such as in the range of about 2.5-3.0.

5. A method according to claim 1, wherein acidification is performed using an inorganic acid, for example hydrochloric acid, e.g. in the form of concentrated hydrochloric acid such as hydrochloric acid having a concentration of about 20-40%.

6. A method according to claim 1, wherein a nanofiltration step is performed using a nanofiltration membrane with the ability to retain divalently charged ions and molecules larger than about 180 g/mol.

7. A method according to claim 1 wherein the isolation of lactic acid further comprises at least one lectrodialysis step.

8. A method according to claim 7 wherein the electrodialysis step is performed by m ans of bipolar electrodialysis m mbranes.

9. A method according to claim 1, wherein the isolation of lactic acid further comprises filtration using activated charcoal.

10. A method according to claim 1, wherein the isolation of lactic acid comprises at least a second nanofiltration and/or reverse osmosis step.

11. A method according to claim 10 further comprising a concentration step wherein the concentration of the lactic acid in the permeate resulting from the nanofiltration step and/or the reverse osmosis step is increased prior to being subjected to said at least second nanofiltration and/or reverse osmosis step.

12. A method according to claim 11 wherein the concentration of the lactic acid is increased to about 5-90%, including about 10-50%, such as about 15-25%, including to about 20%.

13. A method according to claim 1, wherein a protein is present in or is added to the fermentation liquid as a nutrient substrate for the lactic acid-forming bacteria, and wherein at least one protein-hydrolysing enzyme is added to the fermentor during the fermentation so that hydrolysis of protein to amino acids takes place simultaneously with the fermentation of sugar into organic acid.

14. A method according to claim 1 further comprising a concentration step wherein the concentration of the lactic acid in the permeate resulting from said isolation of lactic acid is increased.

15. A method according to claim 14 wherein the concentration of lactic acid is increased to about 50-99%, including about 60-95%, such as about 70-90%.

16. A method for isolating an organic acid from a solution containing an organic acid salt c mprising the steps of:
i) forming a substantially polymer-free permeate containing the organic add salt,
ii) acidifying the permeate to a pH value of below about the pKa-value of the organic acid,
iii) subjecting the acidified permeate to at least one nanofiltration and/or reverse osmosis step to result in a organic acid-containing product,
iv) subjecting the product to an electrodialysis step,
v) concentrating the product of the electrodialysis to result in concentrated organic add, and optionally
vi) polishing the concentrated organic acid, e.g. using nanofiltration or activated charcoal.

17. A method according to claim 16 wherein the organic acid is selected from the group consisting of formic acid, acetic acid, lactic acid, butyric acid, propionic acid, valeric acid, isovaleric acid, capronic acid, heptanoic acid, octanic acid, oxalic add, maloic acid, glutaric acid, adipic acid, glycolic acid, glycinic acid, acrylic acid, tartaric acid, fumaric acid, benzoic acid, maleric add, phthalic acid, and salicylic acid.

18. A method according to claim 16 wherein the organic acid is lactic acid.

19. A method according to claim 18, wherein the permeate is acidified to a pH of below about 3.9, including below about 3.5, such as in the range of about 2.5-3.0.

20. A method according to claim 16 wherein acidification is performed using an inorganic acid, such as hydrochloric acid, e.g. in the form of concentrated hydrochloric acid such as hydrochloric acid having a concentration of about 20-40%.

21. A method according to claim 16, wherein a nanofiltration step is performed using a nanofiltration membrane with the ability to retain divalently charged ions and molecules larger than about 180 g/mol.

22. A method according to claim 16 wherein the electrodialysis step is performed by means of bipolar electrodialysis membranes.

23. A method according to claim 16 comprising at least a second nanofiltration and/or revers osmosis step.

24. A method according to claim 23 further comprising a concentration step wherein th concentration of the organic acid in the permeate resulting from the nanofiltration step and/or the reverse osmosis step is increased prior to being subjected to said at least second nanofiltration and/or reverse osmosis step.

25. A method according to claim 24 wherein the concentration of the organic acid is increased to about 5-90%, including about 10-50%, such as about 15-25%, including to about 20%, prior to being subjected to said at least second nanofiltration and/or reverse osmosis step.

26. A method according to claim 16 wherein the concentration of the concentrated organic acid is in the range of about 50-99%, including about 60-95%, such as about 70-90%.
